(19) United States
(12) Patent Application Publication
(10) Pub. No.: US 2017/0000958 A1
(43) Pub. Date: Jan. 5, 2017

(54) ENTERAL FEEDING WARMING DEVICE AND INTRAVENOUS FLUID WARMING DEVICE

(71) Applicants: SCOTT NORMAN, STILWELL, KS (US); MARK PETHERAM, OVERLAND PARK, KS (US); ANTHONY HASH, OLATHE, KS (US)

(72) Inventors: SCOTT NORMAN, STILWELL, KS (US); MARK PETHERAM, OVERLAND PARK, KS (US); ANTHONY HASH, OLATHE, KS (US)

(21) Appl. No.: 15/269,196

(22) Filed: Sep. 19, 2016

Related U.S. Application Data
(63) Continuation-in-part of application No. 14/262,430, filed on Apr. 25, 2014, now Pat. No. 9,446,205.

(51) Int. Cl. A61M 5/44; A61J 15/00; A61M 5/158

(52) U.S. Cl. CPC A61M 5/445; A61M 5/158; A61J 15/0076; A61M 2205/3368; A61M 2205/502; A61M 2205/583; A61M 2205/3633; A61M 2205/3584; A61M 2205/52

(57) ABSTRACT

An intravenous fluid warming device for warming intravenous fluid and delivering the intravenous fluid to an infant or other patient comprises a housing, a heating element, a heat spreader, a temperature sensor, a controller, a display, a number of user inputs, and a power source. The housing includes outer walls defining an interior chamber and an intravenous tube channel separate from the interior chamber for receiving an intermediate portion of an intravenous tube. The interior chamber retains the heating element, heat spreader, temperature sensor, and controller therein. The intravenous tube channel includes a number of large turns and small turns for increasing an effective length of the intravenous tube channel. The intravenous tube channel is large enough to receive an intravenous tube or an enteral feeding tube. The intravenous fluid warming device may also include a secondary heating element configured to wrap around the intravenous tube.

### RELATED APPLICATIONS

This patent application is a regular utility continuation application and claims priority benefit with regard to all common subject matter of earlier filed non-provisional U.S. patent application Ser. No. 14/262,430, filed on Apr. 25, 2014, and entitled “ENTERAL FEEDING WARMING SYSTEM”. The identified earlier filed non-provisional patent application is hereby incorporated by reference in its entirety into the present application.

### BACKGROUND

The present invention relates to enteral feeding and intravenous fluid warming devices for warming and delivering nutritional fluids and intravenous fluids to infants and other patients.

Infants and other patients who cannot eat through their mouths must be fed nutritional fluids directly into their stomachs or intestines via enteral feeding tubes. Often, such fluids are expressed from a birth mother or prepared in advance and then refrigerated until needed and thus must be warmed prior to feeding. Enteral feeding warming devices have been developed to warm an enteral feeding fluid and to deliver the fluid into an infant's stomach or intestine. These devices typically include a housing and a heating element. The housing encloses the heating element and an intermediate portion of an enteral feeding tube. The enteral feeding tube is then connected to a pump at its first end and is inserted into the infant's stomach or intestine through the infant's esophagus or through an incision in the infant's chest or abdomen at its second end. While fluids are pumped through the enteral feeding tube, they are warmed by the heating device. Similar devices exist for warming intravenous fluids.

The heating elements of enteral feeding warming devices and intravenous fluid warming devices are often heated to a temperature of between 120° F. (49° C.) to 140° F. (60° C.). This high temperature level may negatively break down the nutrients in the fluids such that the infant receives reduced nutritional benefit from the fluids. The high heat may also damage the feeding and/or intravenous fluid tubes and introduce foreign particles from the tubes into the fluids.

### SUMMARY

The present invention solves the above-described problems and provides a distinct advance in the art of enteral feeding warming devices and intravenous fluid warming devices. More particularly, the invention provides an enteral feeding warming device that gradually warms nutritional fluids to a desired temperature and consistently maintains the desired temperature for enterally feeding the fluids to an infant or other patient and an intravenous fluid warming device that gradually warms intravenous fluids to a desired temperature and consistently maintains the desired temperature for intravenously delivering the fluids to an infant or patient.

Applicant has discovered that many patients, and infants in particular, experience feeding tolerance issues unless the refrigerated enteral feeding fluid is warmed to a temperature within a Thermal Neutral Zone (TNZ) between 90° F. (32° C.) and 100° F. (38° C.). Temperatures within this range are ideal for enzymes in the infant's digestive system to break down the nutrients in the enteral feeding fluid. Similarly, temperatures within this range are ideal for the infant's circulatory system to break down nutrients in intravenous fluid.

An embodiment of the invention is an enteral feeding warming device broadly comprising a housing, a heating element, a heat spreader, a temperature sensor, a controller, a display, a number of user inputs, and a power source. The housing includes first and second clamshell housing sections connected together by a hinge or other connecting mechanism. The first clamshell housing section includes outer walls defining an internal heating chamber. One of the outer walls also includes a slot that defines a feeding tube channel extending from one end of the housing to another end of the housing and including a number of turns for increasing its effective length. The turns include retention features such as primary and secondary portions curving in opposite directions in quick succession for retaining an intermediate portion of an enteral feeding tube in the feeding tube channel. The second clamshell housing section includes a mounting component for mounting the warming device onto a stand or other structure.

The heating element may be an electric silicon heating pad or other heating element positioned within the internal heating chamber for warming the enteral feeding fluid. The heat spreader is a metal plate or other heat distribution component and is positioned within the internal heating chamber near the heating element for evenly distributing heat from the heating element to the air in the internal chamber. The temperature sensor is a thermistor or other temperature gauge positioned within the heating chamber for sensing the temperature in the internal heating chamber. The controller includes a printed circuit board (PCB), a memory, and/or other electronic components for controlling the heating element and maintaining a desired temperature of the internal heating chamber. The display is a seven segment LCD display or other display and may include additional LED lights or other indicators for displaying information from the controller. The user inputs are positioned on an outside of the housing of the warming device and may include a power switch, reset button, or other user inputs for operating the warming device. The power source is a power cord or battery and supplies electric power to the heating element, the controller, and the display.

The enteral feeding tube may be any elongated hollow tube connectable to a pump's output at its first end and configured to be inserted into an infant's stomach or intestine at its second end. An intermediate portion of the enteral feeding tube may be inserted into the feeding tube channel of the first clamshell housing section for sending feeding fluid through the warming device.

In use, the enteral feeding warming device heats nutritional fluid to a temperature within the TNZ and delivers the warmed fluid to an infant's stomach or intestine. To prepare the enteral feeding warming device for feeding the infant, a user connects the first end of the enteral feeding tube to a fluid source via the pump's output. The user then positions the intermediate portion of the enteral feeding tube in the feeding tube channel of the first clamshell housing section of the warming device. The user then closes the clamshell housing sections together so that the intermediate portion of the enteral feeding tube is enclosed within the feeding tube channel. The user then plugs in the power source and/or turns on the warming device so that the heating element begins to warm the air inside the internal heating chamber to a temperature between approximately 90° F. (32° C.) and 103° F. (39° C.). The display indicates the temperature of the air inside the internal heating chamber and indicates when the warming device is ready for heating the fluid.

Once the warming device is ready to heat the fluid, the user inserts the second end of the enteral feeding tube into the infant's stomach or intestine via the infant's esophagus or an incision in the infant's abdomen or chest. The user then activates or turns on the pump, which directs fluid from the fluid source into the warming device via the feeding tube. The heating element of the warming device then warms the fluid to a temperature within the TNZ as the fluid passes through the turns of the feeding tube channel of the warming device. The warmed fluid then continues through the feeding tube and into the infant's stomach or intestine.

The controller of the warming device maintains the temperature of the air in the internal heating chamber between approximately 90° F. (32° C.) and 103° F. (39° C.) when the warming device is on. When the controller determines via the temperature sensor that the air inside the internal heating chamber is above 103° F. (39° C.) or is above another predetermined temperature, the controller temporarily deactivates the heating element until the temperature of the air in the internal heating chamber has dropped to another predetermined temperature between 90° F. (32° C.) and 103° F. (39° C.).

Another embodiment of the invention is an intravenous fluid warming device broadly comprising a housing, a heating element, a heat spreader, a temperature sensor, a controller, a display, a number of user inputs, and a power source. The housing may be similar to the housing described above except that the slot defines an intravenous tube channel extending from one end of the housing to another end of the housing and including a number of large turns and small turns for increasing its effective length and retaining an intermediate portion of an intravenous tube in the intravenous tube channel.

The heating element, heat spreader, temperature sensor, controller, display, user inputs, and power source may be similar to the components described above except that the controller is further configured to determine a number of times the intravenous fluid warming device has been turned on and a number of times the intravenous fluid warming device has been used. The display may also indicate the number of times the intravenous fluid warming device has been turned on and the number of times the intravenous fluid warming device has been used.

The intravenous tube may be any elongated hollow tube connectable to a pump's output at its first end and configured to be connected to an infant's vein at its second end via a needle. An intermediate portion of the intravenous tube may be inserted into the intravenous tube channel of the first clamshell housing section for sending intravenous fluid through the intravenous fluid warming device.

Embodiments of the enteral feeding warming device and/or the intravenous fluid warming device described above may also include a secondary heating component, a secondary temperature sensor, and a data port. The secondary heating component extends from the housing for rewarming fluids being delivered through the second end of the tube. The secondary heating component may be a conformable or pre-coiled cord or wire configured to be wrapped around the tube and may be positioned anywhere along the second end of the tube such as adjacent to or spaced from the housing.

The secondary temperature sensor is positioned near the secondary heating component and senses a temperature of the fluid after the secondary heating component has rewarmed the fluid. The data port allows the secondary temperature sensor to be connected to the controller and may be a USB port, eighth inch connector, proprietary connector, or any other electronic port. Alternatively, the temperature sensor may wirelessly communicate with the controller over a radio, near field communication (NFC), Bluetooth, or internet connection.

In use, the controller may temporarily turn the secondary heating component off or lower its output if the temperature of the rewarmed fluid approaches or reaches the temperature of the fluid as it flows from within the internal heating chamber. To that end, a temperature feedback signal may be sent from the temperature sensor to the controller via the data port, thus forming a temperature monitoring feedback loop. This counters some or all of the heat loss of the fluid as it leaves the housing while preventing the fluid from becoming too warm before being delivered to the infant or patient. Alternatively, the secondary heating component may rewarm the fluid to a temperature up to but not greater than an upper limit of the TNZ or any other suitable upper limit, which ensures that the fluid is closest to a maximum allowable temperature before being delivered to the infant or patient.

This summary is provided to introduce a selection of concepts in a simplified form that are further described below in the detailed description. This summary is not intended to identify key features or essential features of the claimed subject matter, nor is it intended to be used to limit the scope of the claimed subject matter. Other aspects and advantages of the invention will be apparent from the following detailed description of the embodiments and the accompanying drawing figures.

### BRIEF DESCRIPTION OF THE DRAWING FIGURES

Embodiments of the invention are described in detail below with reference to the attached drawing figures, wherein:

FIG. 1 is a perspective view of an enteral feeding warming device constructed in accordance with an embodiment of the invention;

FIG. 2 is a rear perspective view of the enteral feeding warming device;

FIG. 3 is a perspective view of the enteral feeding warming device in which the device's clamshell housing sections are shifted to an open position;

FIG. 4 is a partial elevation view of the enteral feeding warming device;

FIG. 5 is an enlarged partial elevation view of the enteral feeding warming device showing the turns of a feeding tube channel in the enteral feeding warming device;

FIG. 6 is another perspective view of the enteral feeding warming device;

FIG. 7 is a schematic view of the electrical components of the enteral feeding warming device;

FIG. 8 is a schematic view of an enteral feeding warming system for use with another embodiment of the invention;

FIG. 9 is a perspective view of an intravenous warming device constructed in accordance with another embodiment of the invention;

FIG. 10 is a rear perspective view of the intravenous warming device of FIG. 9;

FIG. 11 is a perspective view of the intravenous warming device in which the clamshell housing sections are shifted to an open position;

FIG. 12 is a partial elevation view of the intravenous warming device;

FIG. 13 is an enlarged partial elevation view of the turns of the intravenous tube channel of the intravenous warming device;

FIG. 14 is another enlarged perspective view of the intravenous warming device;

FIG. 15 is a schematic view of the electrical components of the intravenous warming device; and

FIG. 16 is a perspective view of a warming device constructed in accordance with yet another embodiment of the invention and including a secondary warming component.

The drawing figures do not limit the invention to the specific embodiments disclosed and described herein. The drawings are not necessarily to scale, emphasis instead being placed upon clearly illustrating the principles of the invention.

### DETAILED DESCRIPTION OF THE EMBODIMENTS

The following detailed description of the invention references the accompanying drawings that illustrate specific embodiments in which the invention can be practiced. The embodiments are intended to describe aspects of the invention in sufficient detail to enable those skilled in the art to practice the invention. Other embodiments can be utilized and changes can be made without departing from the scope of the invention. The following detailed description is, therefore, not to be taken in a limiting sense. The scope of the invention is defined only by the appended claims, along with the full scope of equivalents to which such claims are entitled.

In this description, references to “one embodiment”, “an embodiment”, or “embodiments” mean that the feature or features being referred to are included in at least one embodiment of the technology. Separate references to “one embodiment”, “an embodiment”, or “embodiments” in this description do not necessarily refer to the same embodiment and are also not mutually exclusive unless so stated and/or except as will be readily apparent to those skilled in the art from the description. For example, a feature, structure, act, etc. described in one embodiment may also be included in other embodiments, but is not necessarily included. Thus, the present technology can include a variety of combinations and/or integrations of the embodiments described herein.

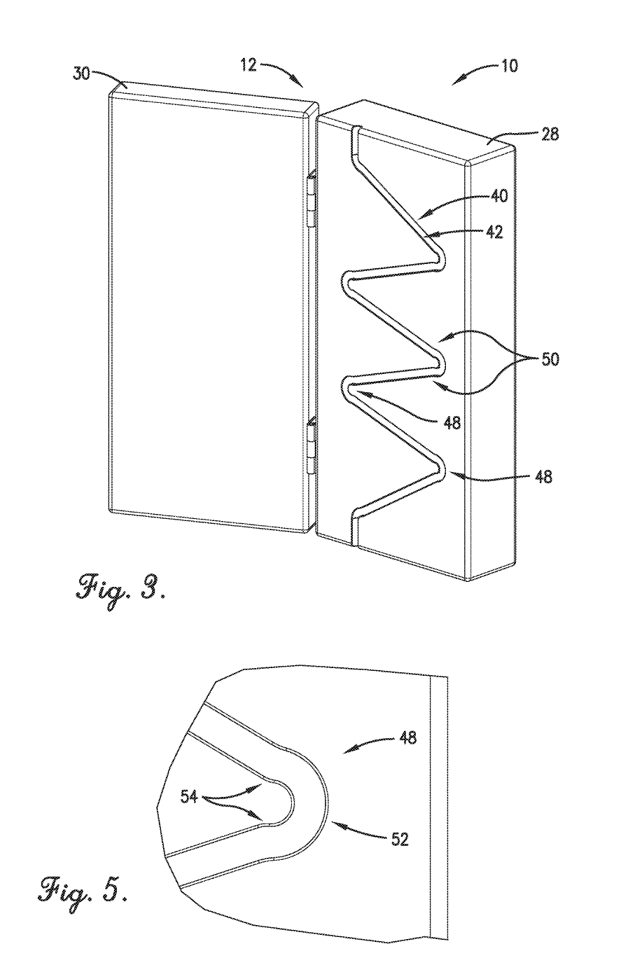

Turning now to FIGS. 1-7, an enteral feeding warming device 10 constructed in accordance with an embodiment of the invention is illustrated. The enteral feeding warming device 10 broadly comprises a housing 12, a heating element 14, a heat spreader 16, a temperature sensor 18, a controller 20, a display 22, one or more user inputs 24, and a power source 26.

The housing 12 encloses and protects the components of the warming device 10 and is formed of water resistant plastic or other heat insulating material. The housing is approximately 7 inches long, 3.25 inches wide, and 2 inches deep and includes first and second clamshell housing sections 28, 30, a set of hinges 32, and a set of magnets 34. The housing 12 together with the above heating components is approximately 22.5 ounces or lighter.

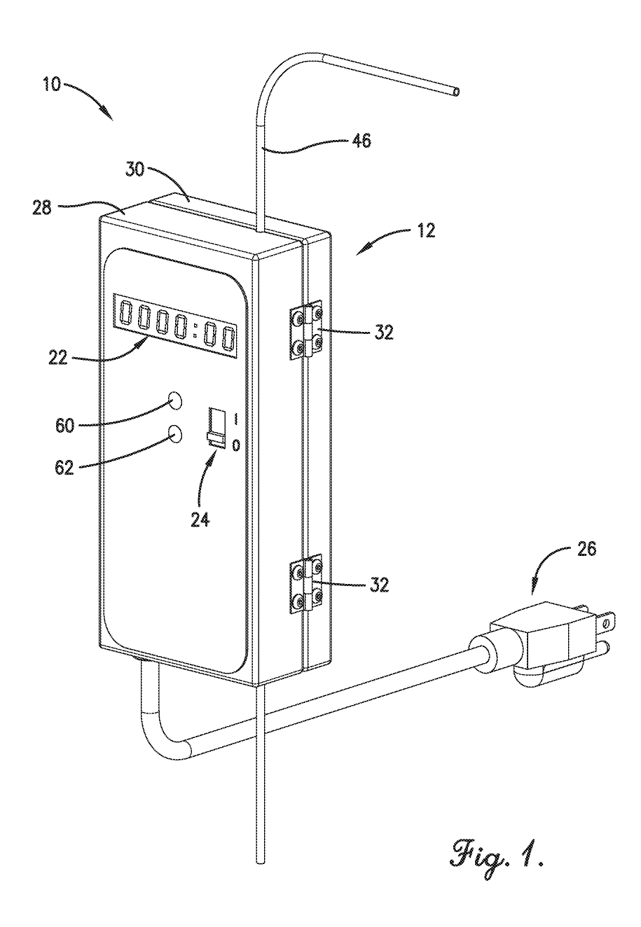

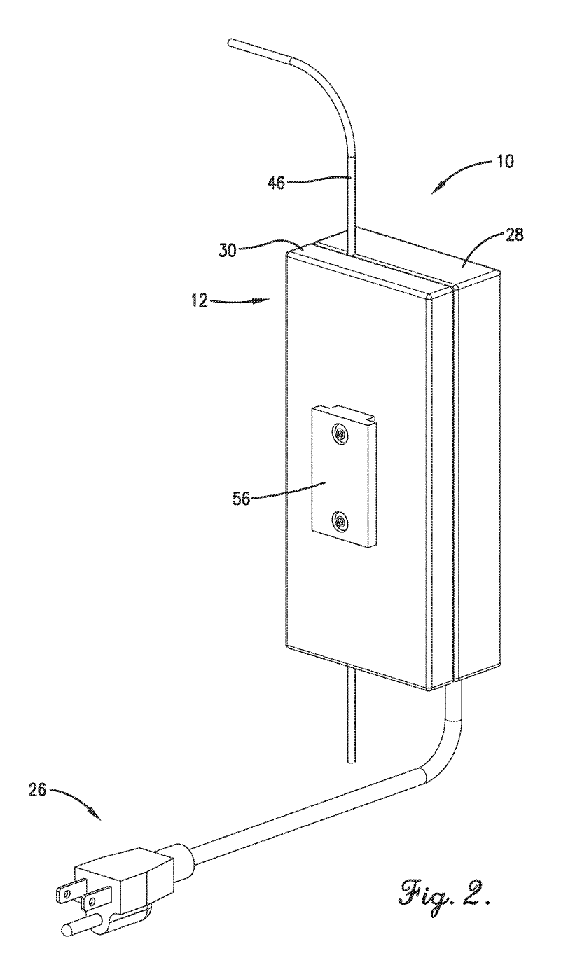

The first clamshell housing section 28 includes outer walls 36 defining an interior chamber 38 and a slot 40 defining a feeding tube channel 42, as shown in FIGS. 3-6. The interior chamber 38 houses the heating element 14, the heat spreader 16, the temperature sensor 18, the controller 20, and the display 22 and includes a front opening 44. The front opening 44 allows the display 22 to be mounted to a front of the first clamshell housing section 28 and allows the display 22 to face outward so that the user can easily view the display 22. The feeding tube channel 42 retains a portion of an enteral feeding tube 46 (described below) within the warming device 10 and is formed into an outer surface of one of the outer walls 36 of the first clamshell housing section 28. The feeding tube channel 42 is approximately 0.06 inches (1.5 mm) to approximately 0.16 inches (4 mm) in diameter and is substantially rounded so as to contact approximately 180° around the outer surface of the enteral feeding tube 46. The feeding tube channel 42 includes five complete turns 48 and six complete straight sections 50 that weave laterally back and forth so that the effective length of the feeding tube channel 42 is approximately 12 inches to approximately 16 inches. The turns 48 include retention features such as primary portions 52 and secondary portions 54 curving in opposite directions in quick succession for retaining the enteral feeding tube 46 in the feeding tube channel 42, as best shown in FIG. 4. The primary portions 52 curve in the direction of the turn. The secondary portions 54 are located on either end of the primary portions 54 and curve in the opposite direction of the turn.

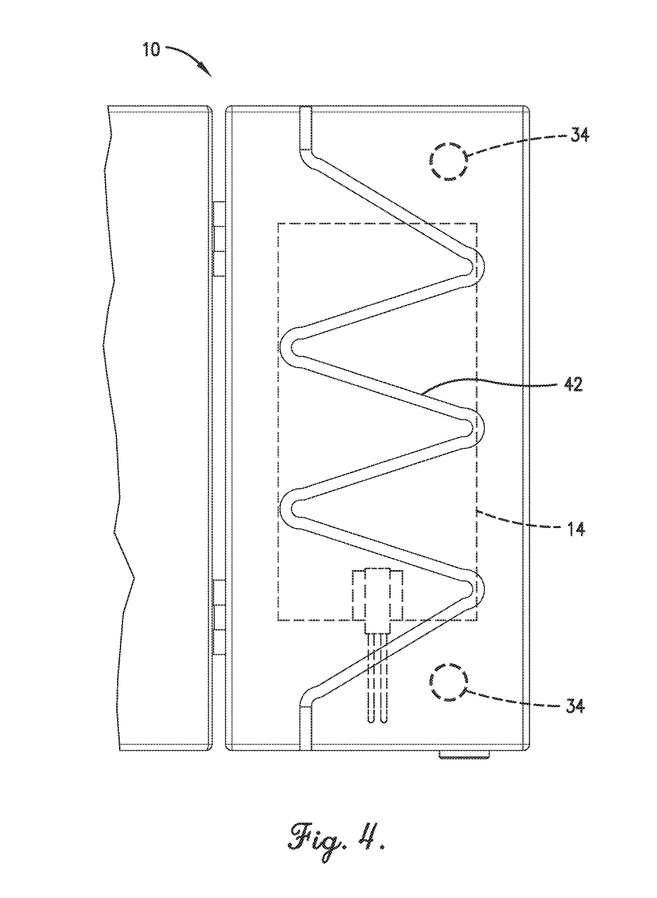

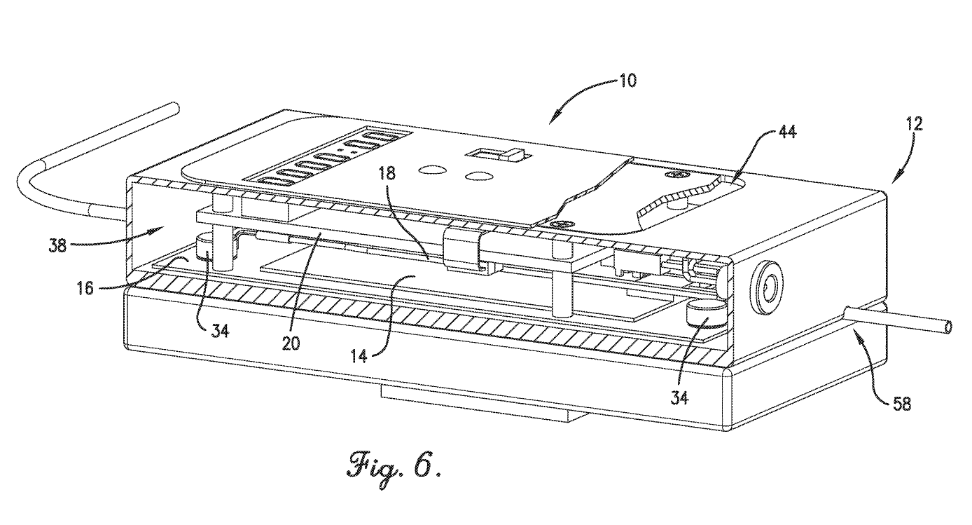

The enteral feeding tube 46 carries the fluid from a pump, through the feeding tube channel 42, and into the infant's stomach or intestine. The enteral feeding tube 46 is plastic or any other suitable material and includes an intermediate portion 58 for inserting into the warming device 10, as shown in FIG. 6. The enteral feeding tube 46 has an outer diameter of approximately 0.06 inches (1.5 mm) to approximately 0.16 inches (4 mm) and an inside diameter sufficiently large to accommodate a fluid flow rate of approximately 0.25 ml/min to approximately 1 ml/min.

The second clamshell housing section 30 encloses the enteral feeding tube 46 in the feeding tube channel 42 when the clamshell housing sections 28, 30 are in a closed position and includes a mounting component 56 positioned on its rear face, as shown in FIG. 2. The mounting component 56 may be a bracket, a hook and loop fastener, a hook, a clip, or any other mounting structure configured to removably attach the housing 12 to a stand, a multi-jointed arm, an incubator, a bed, or other structure.

The hinges 32 allow the clamshell housing sections 28, 30 to be pivoted with respect to each other between a closed position (FIGS. 1 and 2) and an open position (FIG. 3) and are connected to the clamshell housing sections 28, 30 via screws, bolts, rivets, or other fasteners.

The magnets 34 resistively retain the first and second clamshell housing sections 28, 30 in the closed position and are mounted in corresponding locations within or on the first and second clamshell housing sections 28, 30, as shown in FIG. 6. The magnets 34 are attached to the clamshell housing sections 28, 30 via adhesives or mounting bosses formed in the clamshell housing sections 28, 30.

The heating element 14 warms the air in the interior chamber 38 of the first clamshell housing section 28 and is a silicone heating pad, a resistive heater, or other heating element. The heating element 14 is mounted to a back wall of the first clamshell housing section 28 via adhesives or mounting bosses and is connected to the controller 20 and receives electrical power therefrom, as shown in FIG. 6.

The heat spreader 16 evenly spreads the heat generated by the heating element 14 and is a thin metal plate or other heat distribution component formed of aluminum, copper, or other suitable material. The heat spreader 16 is mounted to the back wall of the first clamshell housing section 28 behind the heating element 14 via adhesives or mounting bosses and substantially covers the back wall of the first clamshell housing section 28.

The temperature sensor 18 senses the temperature of the air in the interior chamber 38 of the first clamshell housing section 28 and may be a thermistor, thermocouple, a silicon bandgap temperature sensor, or any other temperature gauge. The temperature sensor 18 is mounted to a wall of the first clamshell housing section 28 in the interior chamber 38 via adhesives or mounting bosses and connected to the controller 20.

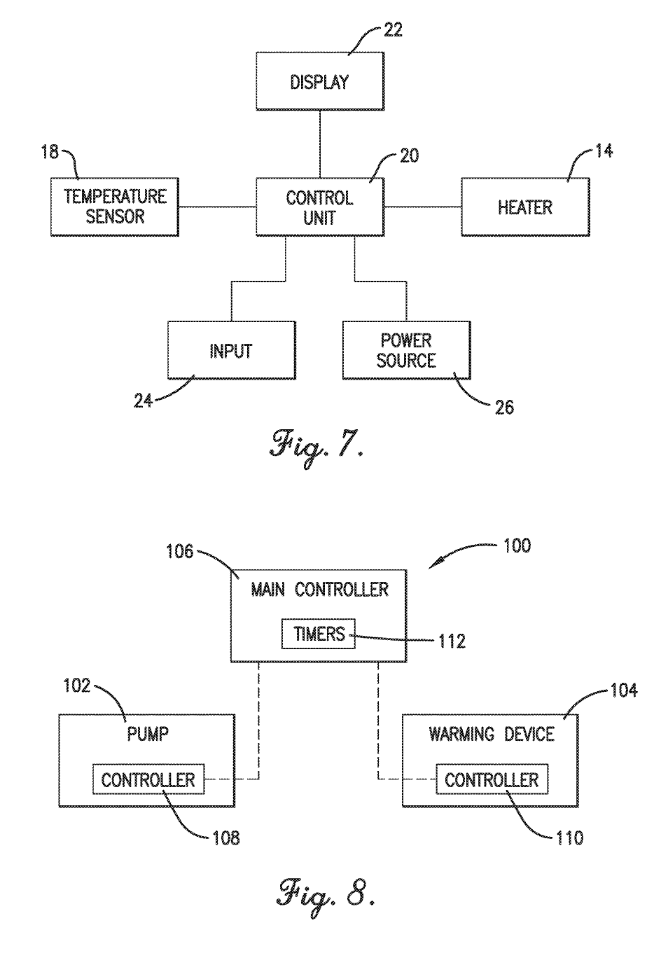

The controller 20 regulates the air temperature in the interior chamber 38 and controls the heating element 14 and the display 22. The controller 20 includes a printed circuit board (PCB), a memory, and/or other electronic components and is mounted to a wall of the first clamshell housing section 28 in the interior chamber 38 via adhesives or mounting bosses. The controller 20 is connected to the heating element 14, the temperature sensor 18, the display 22, the user inputs 24, and the power source 26, as shown in FIG. 7.

The controller 20 may implement aspects of the invention with one or more computer programs stored in or on computer-readable medium residing on or accessible by the controller 20. Each computer program preferably comprises an ordered listing of executable instructions for implementing logical functions in the controller 20. Each computer program can be embodied in any non-transitory computer-readable medium for use by or in connection with an instruction execution system, apparatus, or device, such as a computer-based system, processor-containing system, or other system that can fetch the instructions from the instruction execution system, apparatus, or device, and execute the instructions. In the context of this application, a “computer-readable medium” can be any non-transitory means that can store the program for use by or in connection with the instruction execution system, apparatus, or device. The computer-readable medium can be, for example, but not limited to, an electronic, magnetic, optical, electro-magnetic, infrared, or semi-conductor system, apparatus, or device. More specific, although not inclusive, examples of the computer-readable medium would include the following: an electrical connection having one or more wires, a portable computer diskette, a random access memory (RAM), a read-only memory (ROM), an erasable, programmable, read-only memory (EPROM or Flash memory), an optical fiber, and a portable compact disk read-only memory (CDROM).

The display 22 provides a visual indication of the temperature of the interior chamber 38 and other information and may be a seven segment LCD display, an analog display, a touch screen, or any other display. The display 22 may include additional LED lights 60, 62 and other indicators for providing additional information to the user. The display 22 is mounted in the front opening 44 of the first clamshell housing section 28 facing outward from the housing 12 and connected to the controller, as shown in FIG. 1.

The user inputs 24 allow the user to turn the warming device 10 on and off, to program the warming device 10, to reset the warming device 10, and to perform other functions and may include switches, buttons, dials, and other user inputs. The user inputs 24 may comprise a power button or power switch, a reset button, a calibration button, a temperature unit toggle button, and other inputs.

The power source 26 supplies electrical power to the heating element 14, temperature sensor 18, the controller 20, and the display 22 and includes a power cord for connecting to a 100 volt or 240 volt, 60 W/hr or 100 W/hr, 50/60 Hz outlet, or any other power outlet.

Operation of the enteral feeding warming device 10 will now be described in more detail. First, the enteral feeding tube 46 may be connected to a fluid supply via a pump. The clamshell housing sections 28, 30 of the warming device 10 may then be shifted to an open position. A small prying force may need to be applied to the clamshell housing sections 28, 30 to separate the magnets 34 from each other. The intermediate portion 58 of the enteral feeding tube 46 may then be inserted into the feeding tube channel 42 so that the enteral feeding tube 46 lays flush with the rear face of the first clamshell housing section 28. Small sections of the enteral feeding tube 46 may need to be pushed into the primary and secondary portions 52, 54 of the turns 48 of the feeding tube channel 42 to ensure that the enteral feeding tube 46 is fully seated in the feeding tube channel 42. The clamshell housing sections 28, 30 may then be shifted to a closed position so that the intermediate portion 58 of the enteral feeding tube 46 is fully enclosed in the housing 12 of the warming device 10. The warming device 10 may then be mounted on a stand or other structure via the mounting component 56 or placed in the infant's incubator so that the display 22 is easily readable.

The warming device 10 may then be turned on by plugging in the power source 26 of the warming device 10 or pressing the power button of the user inputs 24. The controller 20 will initiate a power on sequence in which it instructs the display 22 to display an indication that the warming device 10 is fully operational. If the controller 20 or other components of the warming device 10 are not fully operational, the controller 20 may generate an error message and instruct the display 22 to display the error message indicating to the user that the warming device 10 or a component of the warming device 10 should be serviced. The controller 20 will activate the heating element 14 if the warming device 10 is fully functional so that the heating element 14 begins to warm the air in the interior chamber 38 of the first clamshell housing section 28 of the warming device.

The controller 20 monitors the temperature of the air in the interior chamber 38 of the first clamshell housing section 28 by sending a signal to the temperature sensor 18, which returns a signal representative of the temperature to the controller 20. The controller 20 instructs the display 22 to display the temperature of the air in the interior chamber 38. If the air temperature is less than a predetermined lower threshold temperature, such as 90° F. (32° C.), the heating element 14 continues to warm the air in the interior chamber 38 and the controller 20 instructs the display 22 to indicate via the LED light 60 that the warming device 10 is not ready to warm the enteral feeding fluid. Warming the air temperature from room temperature to the predetermined lower threshold temperature should take approximately 2 to 3 minutes.

The controller 20 instructs the display 22 to indicate via the other LED light 62 that the warming device 10 is ready to warm the enteral feeding fluid when the air temperature in the interior chamber 38 reaches the predetermined lower threshold temperature. The second end of the enteral feeding tube 46 may then be inserted into the infant's stomach or intestine and the pump may then be activated. The pump draws the feeding fluid from the fluid supply and forces it through the enteral feeding tube 46 at a flow rate of approximately 0.25 ml/min to approximately 1 ml/min. The warm air in the interior chamber 38 of the first clamshell housing section 28 warms the enteral feeding fluid to a temperature within the TNZ as it passes through the intermediate portion 58 of the enteral feeding tube 46 in the feeding tube channel 42. The warmed enteral feeding fluid continues through the remainder of the enteral feeding tube 46 and is delivered into the infant's stomach or intestine via the second end of the enteral feeding tube 46.

At any time during operation, the controller 20 will temporarily deactivate the heating element 14 if the temperature of the air in the interior chamber 38 is greater than a predetermined upper threshold temperature, such as 103° F. (39° C.). The controller 20 will reactivate the heating element 14 when the air temperature drops to the predetermined lower threshold temperature or when the air temperature drops to an intermediate temperature as described below. This ensures that the temperature of the enteral feeding fluid exiting the intermediate portion 58 of the enteral feeding tube 46 is within the TNZ.

The controller 20 may maintain the temperature of the air in the interior chamber 38 at an intermediate temperature, such as 95° F. (35° C.), or within an intermediate temperature range, such as 93° F. (34° C.) to 97° F. (36° C.), by frequently activating and deactivating the heating element 14 when the air temperature reaches these temperatures. This allows the temperature of the air in the interior chamber 38, and hence the temperature of the enteral feeding fluid exiting the intermediate portion 58 of the enteral feeding tube 46 and being delivered into the infant's stomach or intestine, to remain relatively constant during operation instead of rising and falling between the upper and lower threshold temperatures of the TNZ.

It will be understood that one or more of the above-described steps may be performed in a different order than described or simultaneously. For example, the warming device 10 may be turned on before the first end of the enteral feeding tube 46 is connected to the output of the pump. As another example, the heating element 14 may begin warming the air in the interior chamber 38 of the first clamshell housing section 28 at the same time that the user inserts the intermediate portion 58 of the enteral feeding tube 46 into the interior chamber 38.

The above-described enteral feeding warming device 10 provides several advantages over conventional devices. For example, the warming device 10 gradually warms the enteral feeding fluid to a temperature in the TNZ. The turns 48 and straight sections 50 of the feeding tube channel 42 of the warming device 10 increase the effective length of the feeding tube channel 42, which allows the enteral feeding fluid to be exposed to the heat of the warm air for an extended duration of time. The extended exposure allows the warming device 10 to warm the enteral feeding fluid to a temperature within the TNZ without using high heat to warm the enteral feeding fluid. This prevents the nutrients in the enteral feeding fluid from breaking down and prevents the enteral feeding tube 46 from introducing foreign particles into the fluid. The feeding tube channel 42 being formed on an outside of the first clamshell housing section 28 allows the enteral feeding tube 46 to be inserted into and removed from the warming device 10 without exposing the heating element 14 and the controller 20. The primary and secondary portions 52, 54 of the turns 48 of the feeding tube channel 42 prevent the enteral feeding tube 46 from slipping out of the feeding tube channel 42. The magnets 34 of the first and second clamshell housing sections 28, 30 ensure that the enteral feeding tube 46 is completely enclosed within the warming device 10 so that minimal heat is lost to ambient air. Moreover, the mounting component 56 allows the warming device 10 to be positioned at a convenient height and location so that the display 22 is easily readable and so that the second end of the enteral feeding tube 46 may be easily inserted into the infant's stomach or intestine. The warming device 10 may also be mounted onto or placed inside an incubator for reducing the risk of the warming device 10 becoming dislodged or disconnected during use. The heat spreader 16 also allows the interior chamber 38 of the warming device 10 to be cleaned easily and minimizes the buildup of dirt and debris inside the interior chamber 38.

Turning now to FIG. 8, an enteral feeding warming system 100 constructed in accordance with another embodiment of the invention is illustrated. The enteral feeding warming system 100 comprises a pump 102, a warming device 104, a main controller 106, and an enteral feeding tube.

The pump 102 includes a controller 108 that controls the pump 102 and communicates with the main controller 106. The controller 108 includes electronic components similar to the controller described above and may include a transceiver for sending wireless signals to and receiving wireless signals from the main controller 106.

The warming device 104 is substantially similar to the warming device described above except the warming device 104 includes a controller 110 that controls the warming device 104 and communicates with the main controller 106. The controller 110 includes electrical components similar to the controllers described above and may include a transceiver for sending wireless signals to and receiving wireless signals from the main controller 106.

The main controller 106 is substantially similar to the controllers described above except the controller 106 includes one or more timers 112, a transceiver, and other electronic components. The timers 112 trigger controller operations based on calculated or pre-determined time constraints and may be quartz timers or simulated timers.

The main controller 106 coordinates operation of the pump 102 and the warming device 104 via the pump controller 108 and the warming device controller 110 to regulate fluid flow rate and fluid temperature. For example, to increase the fluid flow rate of the fluid being fed to the infant, the main controller 106 transmits a signal representing an instruction to increase pump speed to the pump controller 108. The pump controller 108 receives the signal via its transceiver and instructs the pump 102 to increase its speed, thereby increasing the fluid flow rate of the fluid being fed to the infant. The main controller 106 also transmits a signal representing an instruction to increase a temperature of the warming device 104 to the warming device controller 110 to compensate for the reduction of warming time of the fluid passing through the warming device 104. The warming device controller 110 receives the signal via its transceiver and instructs the heating element of the warming device 104 to increase the temperature within the internal chamber of the warming device 104. The increased temperature warms the faster-moving fluid passing through the warming device 104 to a temperature substantially the same as the temperature reached by the slower-moving fluid. To decrease the fluid flow rate of the fluid being fed to the infant, the main controller 106 instructs the pump controller 108 to decrease the pump speed, thereby decreasing the fluid flow rate. The main controller 106 also instructs the warming device 104 to decrease its internal temperature to compensate for the increase in warming time of the fluid passing through the warming device 104.

The controllers 106, 108, and 110 may perform the above operations at timed intervals using the timer 112. For example, the main controller 106 may instruct the warming device 104 to increase or decrease its internal temperature and simultaneously start the timer 112 before activating the pump 102 so that the heating element of the warming device 104 has time to warm up. Once the timer 112 reaches a predetermined time interval such as ten seconds to three minutes, the timer 112 may trigger the main controller 106 to instruct the pump 102 to increase or decrease its fluid flow rate.

It will be understood that one or more of the above-described steps may be performed in a different order than described or simultaneously.

The above-described enteral feeding warming system 100 provides several advantages over conventional systems. For example, the enteral feeding warming system 100 may increase and decrease the fluid flow rate and the temperature of the fluid without increasing or decreasing the other. The enteral feeding warming system 100 may also fine-tune the fluid flow rate and the temperature of the fluid for each infant's medical needs. In addition, timing of the above operations may be modified as needed via the timer 112 of the main controller 106.

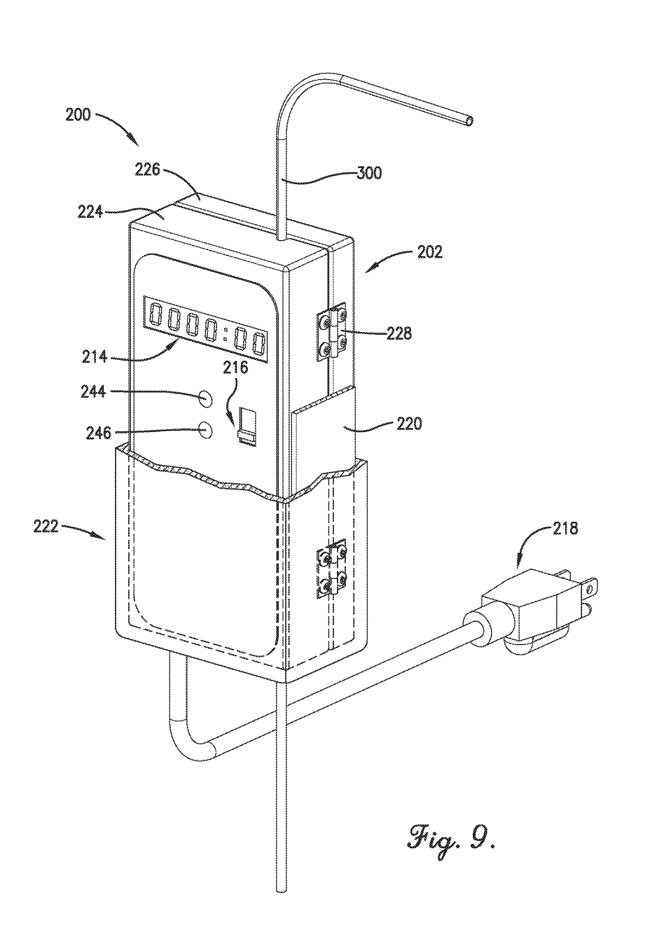

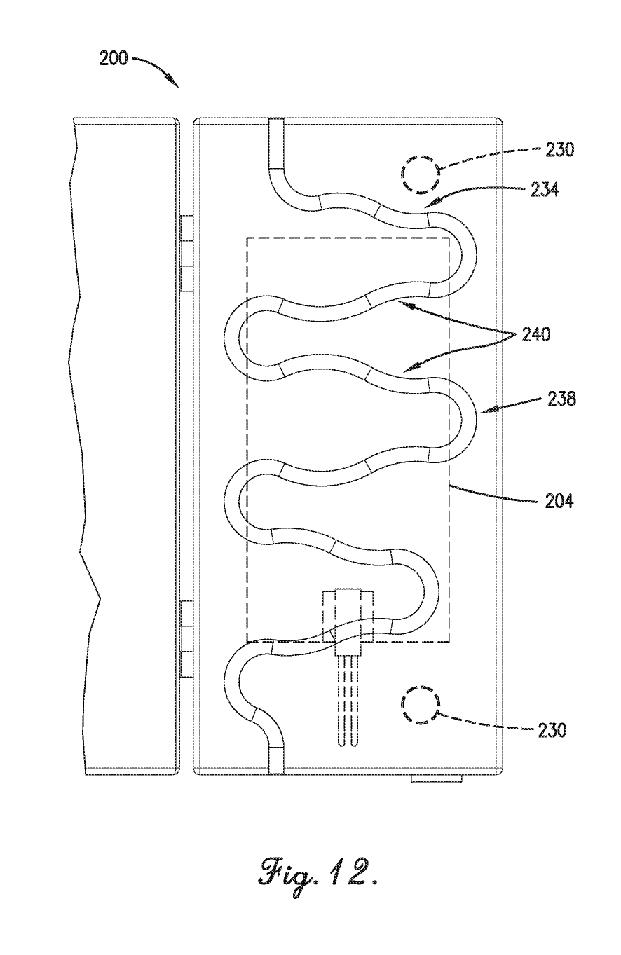

Turning to FIGS. 9-15, an intravenous fluid warming device 200 constructed in accordance with yet another embodiment of the invention for use with an intravenous tube 300 is illustrated. The intravenous fluid warming device 200 broadly comprises a housing 202, a heating element 204, a heat spreader 206, a temperature sensor 208, a controller 210, a transceiver 212 a display 214, one or more user inputs 216, and a power source 218. The intravenous fluid warming device 200 may also include insulating material 220 and/or a cover 222.

The housing 202 encloses and protects the components of the warming device 200 and may be formed of water resistant plastic or other heat insulating material. The housing 202 may be approximately 7 inches long, 3.25 inches wide, and 2 inches deep and includes first and second clamshell housing sections 224, 226, a set of hinges 228, and a set of magnets 230. The housing 202 together with the above heating components may be approximately 22.5 ounces or lighter.

The first clamshell housing section 224 includes outer walls 232 defining an interior chamber 232 and an intravenous tube channel 234, as shown in FIGS. 11-14. The interior chamber 232 houses the heating element 204, the heat spreader 206, the temperature sensor 208, the controller 210, and the display 214 and includes a front opening 236. The front opening 236 allows the display 214 to be mounted to a front of the first clamshell housing section 224 and allows the display 214 to face outward so that the user can easily view the display 214. The intravenous tube channel 234 retains the intravenous tube 300 or an enteral feeding tube within the warming device 200 and is formed into an outer surface of one of the outer walls 232 of the first clamshell housing section 224. The intravenous tube channel 234 may be approximately 0.06 inches (1.5 mm) to approximately 0.16 inches (4 mm) in diameter and may be substantially rounded so as to contact approximately 180° around the outer surface of the enteral feeding tube 46. The intravenous tube channel 234 may have a sufficiently large diameter (e.g., wider and deeper) for receiving the intravenous tube 300. The intravenous tube channel 234 may include five small curves 238 and six large curves 240 that weave laterally back and forth so that the effective length of the intravenous tube channel 234 is approximately 12 inches to approximately 16 inches. The small curves 238 may reverse direction at an effective angle of twelve degrees. The large curves 240 have greater radii than the small curves 238 and may have effective angles (from a straight line) of between thirty degrees and forty-eight degrees. In one embodiment, the large curves 240 have effective angles of thirty-seven degrees. The small curves 238 and large curves 240 may extend back-to-back such that the intravenous tube channel 234 continuously curves through the housing 202. The gradual curvature of the intravenous tube channel 234 may prevent kinking and/or pinching of the intravenous tube 300 while maximizing heat transfer within the warming device 200.

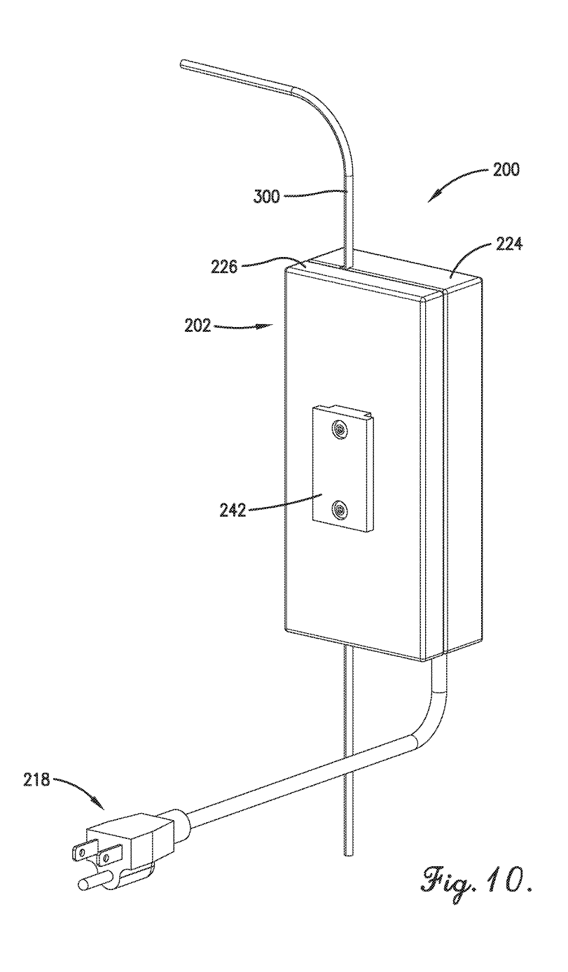

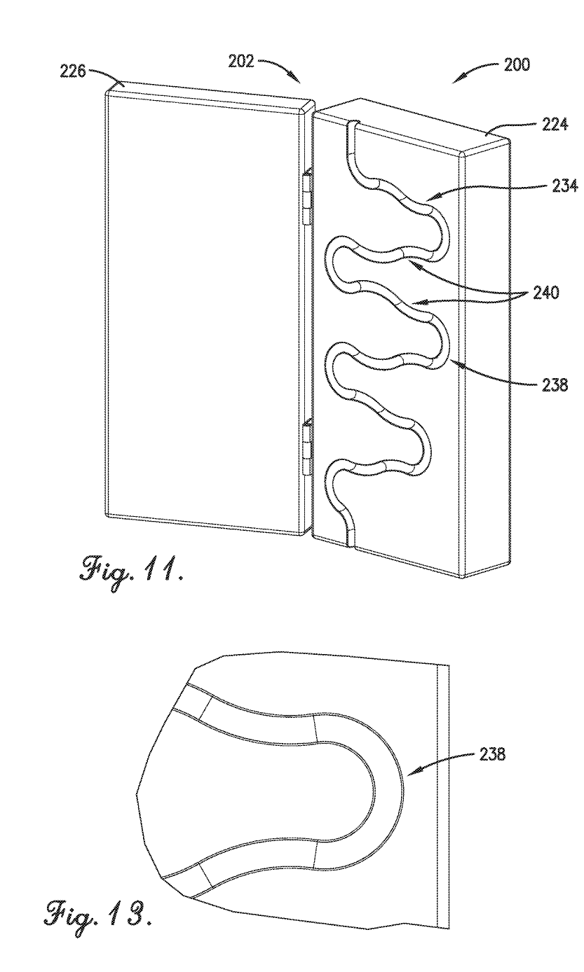

The second clamshell housing section 226 encloses the intravenous tube 300 in the intravenous tube channel 234 when the clamshell housing sections 224, 226 are in a closed position and includes a mounting component 242 positioned on its rear face, as shown in FIG. 10. The mounting component 242 may be a bracket, a hook and loop fastener, a hook, a clip, or any other mounting structure configured to removably attach the housing 202 to a stand, a multi-jointed arm, an incubator, a bed, or other structure.

The hinges 228 allow the clamshell housing sections 224, 226 to be pivoted with respect to each other between a closed position (FIGS. 9 and 10) and an open position (FIG. 11) and are connected to the clamshell housing sections 224, 226 via screws, bolts, rivets, or other fasteners.

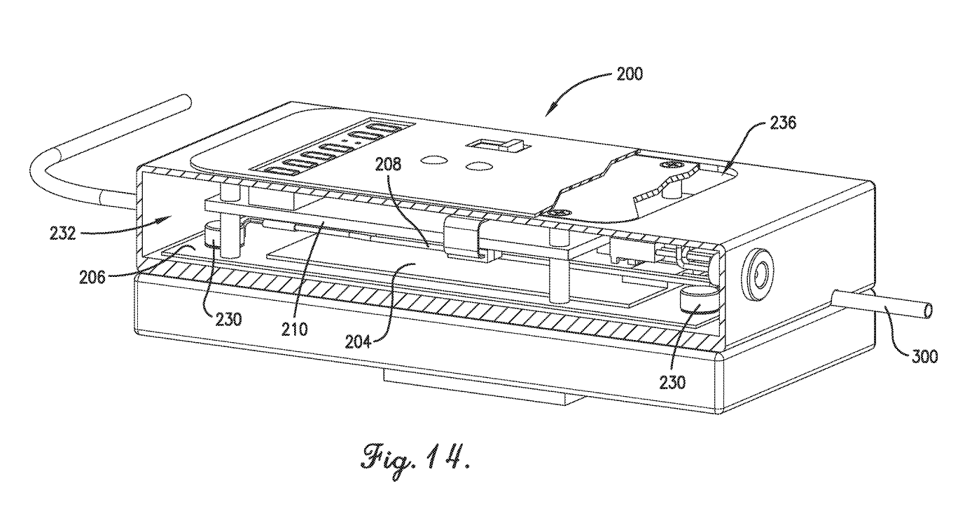

The magnets 230 resistively retain the first and second clamshell housing sections 224, 226 in the closed position and are mounted in corresponding locations within or on the first and second clamshell housing sections 224, 226, as shown in FIG. 14. The magnets 230 are attached to the clamshell housing sections 224, 226 via adhesives or mounting bosses formed in the clamshell housing sections 224, 226.

The heating element 204 warms the air in the interior chamber 232 of the first clamshell housing section 224 and is a silicone heating pad, a resistive heater, or other heating element. The heating element 204 is mounted to a back wall of the first clamshell housing section 224 via adhesives or mounting bosses and may be connected to the controller 210 and receives electrical power therefrom, as shown in FIG. 14. The heating element 204 may be configured to warm intravenous fluids to body temperature (approximately 98.6 degrees Fahrenheit).

The heat spreader 206 evenly spreads the heat generated by the heating element 204 and is a thin metal plate or other heat distribution component formed of aluminum, copper, or other suitable material. The heat spreader 206 may be mounted to the back wall of the first clamshell housing section 224 behind the heating element 204 via adhesives or mounting bosses and substantially covers the back wall of the first clamshell housing section 224.

The temperature sensor 208 senses the temperature of the air in the interior chamber 232 of the first clamshell housing section 224 and may be a thermistor, thermocouple, a silicon bandgap temperature sensor, or any other temperature gauge. The temperature sensor 208 may be mounted to a wall of the first clamshell housing section 224 in the interior chamber 232 via adhesives or mounting bosses and connected to the controller 210.

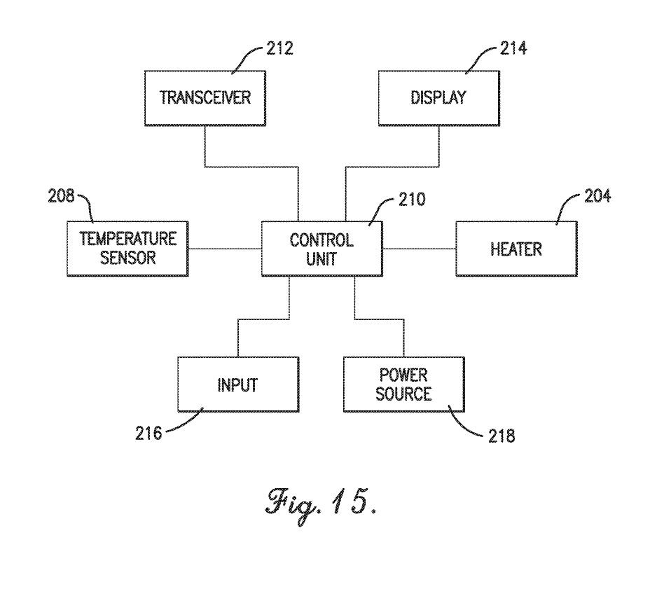

The controller 210 regulates the air temperature in the interior chamber 232 and controls the heating element 204 and the display 214. The controller 210 includes a printed circuit board (PCB), a memory, and/or other electronic components and may be mounted to a wall of the first clamshell housing section 224 in the interior chamber 232 via adhesives or mounting bosses. The controller 210 may be connected to the heating element 204, the temperature sensor 208, the display 214, the user inputs 216, and the power source 218, as shown in FIG. 15. The controller 210 may be configured to count a number of times the intravenous fluid warming device 200 has been used and count the number of times the intravenous fluid warming device 200 has been turned on (i.e., usage information). This will allow a user to determine whether the intravenous fluid warming device 200 may need maintenance or may need replacement parts.

The controller 210 may implement aspects of the invention with one or more computer programs stored in or on computer-readable medium residing on or accessible by the controller 210. Each computer program preferably comprises an ordered listing of executable instructions for implementing logical functions in the controller 210. Each computer program can be embodied in any non-transitory computer-readable medium for use by or in connection with an instruction execution system, apparatus, or device, such as a computer-based system, processor-containing system, or other system that can fetch the instructions from the instruction execution system, apparatus, or device, and execute the instructions. In the context of this application, a “computer-readable medium” can be any non-transitory means that can store the program for use by or in connection with the instruction execution system, apparatus, or device. The computer-readable medium can be, for example, but not limited to, an electronic, magnetic, optical, electro-magnetic, infrared, or semi-conductor system, apparatus, or device. More specific, although not inclusive, examples of the computer-readable medium would include the following: an electrical connection having one or more wires, a portable computer diskette, a random access memory (RAM), a read-only memory (ROM), an erasable, programmable, read-only memory (EPROM or Flash memory), an optical fiber, and a portable compact disk read-only memory (CDROM).

The transceiver 212 transmits data between the controller 210 and external computing devices and may be a Bluetooth antenna or any other suitable transceiver for transmitting data over a wireless communication network. The transceiver 212 may transmit the usage information, current temperature or status, or any other information to the external computing devices.

The display 214 provides a visual indication of the temperature of the interior chamber 232 and other information and may be a seven segment LCD display, an analog display, a touch screen, or any other display. The display 214 may include additional LED lights 244, 246 and other indicators for providing additional information to the user. The display 214 is mounted in the front opening 236 of the first clamshell housing section 224 facing outward from the housing 202 and connected to the controller, as shown in FIG. 9.

The user inputs 216 allow the user to turn the warming device 200 on and off, to program the warming device 200, to reset the warming device 200, and to perform other functions and may include switches, buttons, dials, and other user inputs. The user inputs 216 may comprise a power button or power switch, a reset button, a calibration button, a temperature unit toggle button, and other inputs. The user inputs 216 may also allow the user to instruct the warming device 200 to transmit usage information or other data to the external computing devices or to manage the data locally on the warming device 200.

The power source 218 supplies electrical power to the heating element 204, temperature sensor 208, the controller 210, and the display 214 and includes a power cord for connecting to a 100 volt or 240 volt, 60 W/hr or 100 W/hr, 50/60 Hz outlet, or any other power outlet.

The insulating material 220 provides additional insulation to the intravenous fluid warming device 200 and may be self-adhesive thermal insulating tape or any other suitable insulating material. In one embodiment, the insulating material 220 may be insulating tape configured to adhere to sides of the intravenous fluid warming device 200 over the adjacent edges of the clamshell housing sections 224, 226. This helps prevent warm air from escaping between the clamshell housing sections 224, 226.

The cover 222 provides additional insulation and protection to the intravenous fluid warming device 200 and may be a case or sleeve formed of insulation material. The cover 222 may enclose the intravenous fluid warming device 200 via a zipper, a seal, button snaps, or other suitable fasteners for increasing the insulation effect of the cover 222.

The intravenous tube 300 carries blood, medicine, or other fluids through the intravenous tube channel 234 and into the infant's veins. The intravenous tube 300 may be substantially similar to the enteral feeding tube described above except the intravenous tube 300 may have a smaller or larger outer diameter than the enteral feeding tube, such as an outer diameter of between approximately 0.03 inches (0.7 mm) to approximately 0.32 inches (8 mm). For example, the intravenous tube 300 may have an outer diameter of approximately 0.06 inches (1.5 mm) to approximately 0.16 inches (4 mm) and an inside diameter sufficiently large to accommodate a fluid flow rate of approximately 0.25 ml/min to approximately 1 ml/min. The intravenous tube 300 may be plastic or any other suitable material and includes an intermediate portion for inserting into the warming device 200 into the intravenous tube channel 234, as shown in FIG. 14.

The intravenous fluid warming device 200 may be used in substantially the same way as the enteral feeding warming devices 14, 104 described above. That is, the intravenous fluid warming device 200 may be activated until the air in the interior chamber 232 is sufficiently warm enough to heat the intravenous fluid or other fluid as it passes through the intravenous warming device 200. The controller 210 may cycle the heating element 204 so that the fluid temperature remains within the TNZ.

The above-described intravenous fluid warming device 200 provides several advantages over conventional intravenous fluid warming devices. For example, the gradual curvature of the intravenous tube channel 234 prevents kinking and/or pinching of the enteral feeding tube while maximizing heat transfer within the intravenous warming device 200. The intravenous fluid warming device 200 may also be used for warming enteral feeding fluids. The intravenous fluid warming device 200 may also keep track of the number of times it has been used and/or turned on.

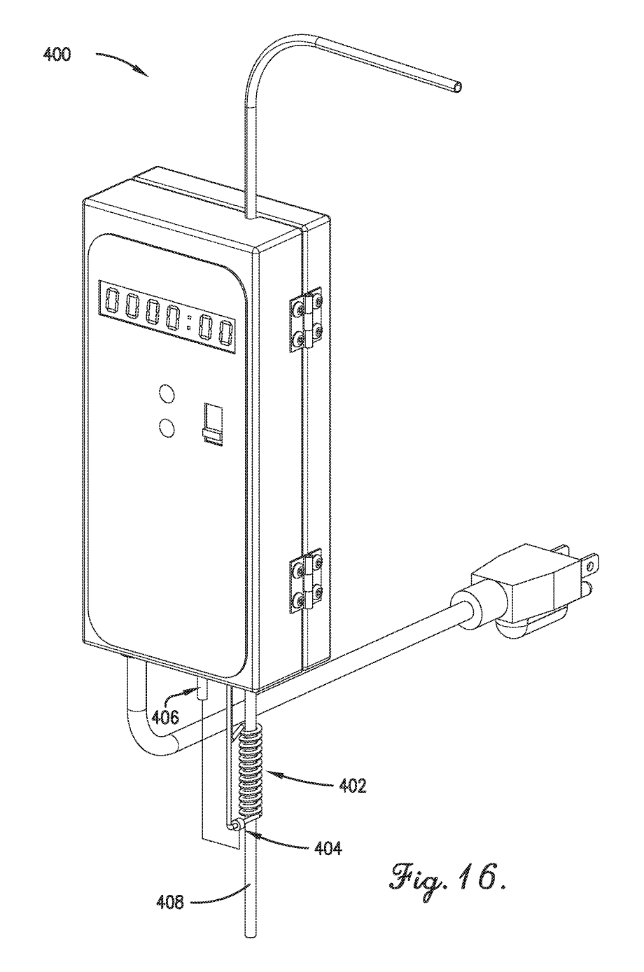

FIG. 16 illustrates a warming device 400 constructed in accordance with another embodiment of the invention. The warming device 400 is substantially similar to the enteral feeding warming device 10 and the intravenous fluid warming device 200 in that the warming device 400 broadly comprises a housing, a heating element, a heat spreader, a temperature sensor, a controller, a display, one or more user inputs, and a power source. The warming device 400 further comprises a secondary heating component 402, a secondary temperature sensor 404, and a data port 406.

The secondary heating component 402 rewarms the fluid after it flows from the housing of the warming device 400 to the second end of the tube 408. The secondary heating component 402 may be a conformable cord or wire (having a protective or non-conductive sheath) configured to be coiled around the tube 408 and may extend and be connected to the housing of the warming device 400 for being powered jointly with the other electronic components of the secondary heating component 402. Alternatively, the secondary heating component 402 may have a preformed coil for allowing the secondary heating component 402 to naturally wrap around the tube 408. The secondary heating component 402 may instead be a clip-on or otherwise attachable component having its own housing. The secondary heating component 402 may be positioned anywhere along the tube 408, such as adjacent to the housing of the warming device 400 or adjacent to the second end of the tube.

The secondary temperature sensor 404 may be a thermistor or other temperature gauge positioned on or near the secondary heating component 402 or in or on the tube 408 near the secondary heating component 402. The secondary temperature sensor 404 senses a temperature of the fluid while or after the fluid is rewarmed by the secondary heating component 402.

The data port 406 is accessible from an outer surface of the housing and allows the secondary temperature sensor 404 to be connected to the controller. The data port 406 may be a USB port, eighth inch connector, proprietary connector, or any other electronic port. Alternatively, the temperature sensor 404 may wirelessly communicate with the controller over a radio, near field communication (NFC), Bluetooth, or internet connection.

In use, the secondary heating component 402 rewarms the fluid to or retains the fluid at a temperature up to but not greater than a temperature of the fluid as it flows out of the housing of the warming device 400. To that end, a feedback signal representative of the temperature sensed by the secondary temperature sensor 404 may be sent to the controller of the warming device 400 via the data port 406, thus forming a temperature monitoring feedback loop. The controller may temporarily turn off or lower the output of the secondary heating component 402 when the feedback signal indicates that the temperature sensed by the secondary temperature sensor 404 is approaching or equal to the temperature of the fluid after the fluid was warmed by the heating element. The controller may also turn on or increase the output of the secondary heating component 402 when the feedback signal indicates that the temperature sensed by the secondary temperature sensor 404 is not approaching or is not equal to the temperature of the fluid after the fluid was warmed by the heating element. This counters some or all of the heat loss of the fluid after it leaves the housing while preventing the fluid from becoming too warm before being delivered to the infant or patient.

Alternatively, the secondary heating component 402 may rewarm the fluid to a temperature up to but not greater than an upper limit of the TNZ or any other suitable upper limit. For example, the controller may temporarily turn off or lower the output of the secondary heating component 402 when the feedback signal indicates that the temperature sensed by the secondary temperature sensor 404 is approaching or equal to the upper limit of the TNZ. This ensures that the fluid is closest to a maximum allowable temperature before being delivered to the infant or patient.

Although the invention has been described with reference to the embodiments illustrated in the attached drawing figures, it is noted that equivalents may be employed and substitutions made herein without departing from the scope of the invention as recited in the claims.

Having thus described various embodiments of the invention, what is claimed as new and desired to be protected by Letters Patent includes the following:

1. An intravenous fluid warming device comprising:
a housing;
a channel positioned in the housing for receiving an intravenous tube having a first end for receiving intravenous fluid from a storage source and a second end for administering the intravenous fluid to a patient;
a heating element positioned within the housing and configured to heat the channel for warming the intravenous fluid in the intravenous tube;
a heat spreader incorporated in the housing for uniformly spreading heat from the heating element;
a temperature sensor for sensing a temperature of the internal heating chamber;
a display configured to indicate an internal temperature of the intravenous fluid warming device, the display being further configured to indicate when the intravenous fluid warming device is ready to begin warming the intravenous fluid for being administered to the patient; and
a controller for controlling operation of the heating element based on temperature readings received from the temperature sensor such that the intravenous fluid administered from the second end of the intravenous tube is warmed to an optimal heating range, the controller being configured to determine a number of times the intravenous fluid warming device has been used and a number of times the intravenous fluid warming device has been turned on and indicate the number of times the intravenous fluid warming device has been used and the number of times the intravenous fluid warming device has been turned on via the display.

2. The intravenous fluid warming device of claim 1, wherein the channel comprises a number of curves for increasing the effective length of the channel.

3. The intravenous fluid warming device of claim 2, wherein the curves include a number of large curves and a number of small curves between the large curves.

4. The intravenous fluid warming device of claim 3, wherein the large curves have an effective angle of between 30 degrees and 48 degrees.

5. The intravenous fluid warming device of claim 4, wherein the large curves each have an effective angle of 37 degrees.

6. The intravenous fluid warming device of claim 4, wherein the small curves each have an effective angle of 12 degrees.

7. The intravenous fluid warming device of claim 4, wherein the small curves each form a turn greater than 180 degrees.

8. The intravenous fluid warming device of claim 2, wherein the curves extend end-to-end so that the channel continuously curves back and forth.

9. The intravenous fluid warming device of claim 1, wherein the channel is further configured to receive an enteral feeding tube.

10. The intravenous fluid warming device of claim 1, further comprising self-adhesive thermal insulating material configured to be positioned around the housing for insulating the housing and the intravenous tube.

11. The intravenous fluid warming device of claim 1, further comprising a cover formed of thermal insulating material, the housing being configured to be placed in the cover for insulating the housing and the intravenous tube.

12. The intravenous fluid warming device of claim 1, further comprising a transceiver configured to transmit the number of times the intravenous fluid warming device has been used and the number of times the intravenous fluid warming device has been used to an external device over a wireless communication network.

13. An intravenous fluid warming device comprising:
a housing;
a channel positioned in the housing for receiving an intravenous tube, the channel comprising a number of curves for increasing an effective length of the channel, the curves including a number of large curves and a number of small curves, the intravenous tube having a first end for receiving intravenous fluid from a storage source and a second end for administering the intravenous fluid to a patient;
a heating element positioned within the housing and configured to heat the channel for warming the intravenous fluid in the intravenous tube;
a heat spreader incorporated in the housing for uniformly spreading heat from the heating element;
a temperature sensor for sensing a temperature of the internal heating chamber;
a display configured to indicate an internal temperature of the intravenous fluid warming device, the display being further configured to indicate when the intravenous fluid warming device is ready to begin warming the intravenous fluid for being administered to the patient;
a controller for controlling operation of the heating element based on temperature readings received from the temperature sensor such that the intravenous fluid administered from the second end of the intravenous tube is warmed to an optimal heating range;
a secondary heating component configured to be positioned near the intravenous tube between the housing of the intravenous fluid warming device and the second end of the intravenous tube for rewarming the intravenous fluid as the intravenous fluid passes from the housing of the intravenous fluid warming device to the second end of the intravenous tube;
a secondary temperature sensor configured to be positioned near the secondary heating component for sensing a temperature of the intravenous fluid after the intravenous fluid has been heated by the secondary heating component;
a data port for receiving a feedback loop signal from the secondary temperature sensor, the feedback loop signal corresponding to the temperature of the intravenous fluid as sensed by the secondary temperature sensor, the controller being configured to adjust an output of the secondary heating component based on the feedback loop signal.

14. The intravenous fluid warming device of claim 13, wherein the secondary heating component is a conformable electric cord configured to be coiled around the intravenous tube.

15. The intravenous fluid warming device of claim 14, wherein the secondary heating component extends from the housing of the intravenous fluid warming device.

16. The intravenous fluid warming device of claim 14, wherein the secondary heating component is configured to warm the intravenous fluid to no more than a temperature of the intravenous fluid exiting the housing of the intravenous fluid warming device.

17. The intravenous fluid warming device of claim 13, wherein the curves extend end-to-end so that the channel continuously curves back and forth.

18. The intravenous fluid warming device of claim 13, wherein the small curves each form a turn greater than 180 degrees.

19. The intravenous fluid warming device of claim 13, wherein the channel is further configured to receive an enteral feeding tube.

20. An intravenous fluid warming device comprising:
a housing;
a channel positioned in the housing for receiving an intravenous tube, the channel comprising six large curves and five small curves between the large curves for increasing the effective length of the channel, the large curves having an effective angle of between 30 degrees and 48 degrees, the small curves having an effective angle of at least 12 degrees, the large curves and the small curves extending end-to-end so that the channel continuously curves back and forth, the intravenous tube having a first end for receiving intravenous fluid from a storage source and a second end for administering the intravenous fluid to a patient;
a heating element positioned within the housing and configured to heat the channel for warming the intravenous fluid in the intravenous tube;
a heat spreader incorporated in the housing for uniformly spreading heat from the heating element;
a temperature sensor for sensing a temperature of the internal heating chamber;
a display configured to indicate an internal temperature of the intravenous fluid warming device, the display being further configured to indicate when the intravenous fluid warming device is ready to begin warming the intravenous fluid for being administered to the patient;
a controller for controlling operation of the heating element based on temperature readings received from the temperature sensor such that the intravenous fluid administered from the second end of the intravenous tube is warmed to an optimal heating range the controller being configured to determine a number of times the intravenous fluid warming device has been used and a number of times the intravenous fluid warming device has been turned on and indicate the number of times the intravenous fluid warming device has been used and the number of times the intravenous fluid warming device has been turned on via the display; and
a cover formed of thermal insulating material, the housing being configured to be placed in the cover for insulating the housing and the intravenous tube.
